(19) United States
(12) Patent Application Publication
(10) Pub. No.: US 2024/0306901 A1
(43) Pub. Date: Sep. 19, 2024

(54) MEDICAL DEVICES INCLUDING ARTICULATION JOINTS AND ASSOCIATED METHODS OF ASSEMBLY

(71) Applicant: Boston Scientific Scimed, Inc., Maple Grove, MN (US)

(72) Inventors: Abdiqadar Ahmed MOHAMUD, Coon Rapids, MN (US); Brent DOLAN, Minneapolis, MN (US); Brian Paul EDISON, Otsego, MN (US); Steven LUNDEEN, Ramsey, MN (US)

(73) Assignee: Boston Scientific Scimed, Inc., Maple Grove, MN (US)

(21) Appl. No.: 18/604,606

(22) Filed: Mar. 14, 2024

Related U.S. Application Data
(60) Provisional application No. 63/490,784, filed on Mar. 17, 2023.

(51) Int. Cl. A61B 1/008; A61B 1/00; A61B 1/005; A61M 25/01

(52) U.S. Cl. CPC A61B 1/008; A61B 1/0011; A61B 1/0055; A61B 1/0057; A61M 25/0138; A61M 25/0147

(57) ABSTRACT

An articulation joint for a medical device has a proximal end and a distal end. The articulation joint includes a plurality of links, one or more actuating wire, and at least one stiffening element. Each link of the plurality of links is configured to articulate relative to adjacent links of the plurality of links. Each actuating wire extends through an actuation channel of the plurality of links. Each actuating wire is fixedly attached proximate the distal end of the articulation joint and translatable relative to the plurality of links to articulate the articulation joint. The at least one stiffening element extends through a stiffening channel of the plurality of links. The at least one stiffening element has a proximal end fixedly attached to one link of the plurality of links and has a distal end fixedly attached to another link of the plurality of links.

### CROSS-REFERENCE TO RELATED APPLICATIONS

This application claims the benefit of priority under 35 U.S.C. § 119 from U.S. Provisional Application No. 63/490,784, filed Mar. 17, 2023, which is incorporated by reference herein in its entirety.

### TECHNICAL FIELD

This disclosure relates generally to medical devices including articulation joints and methods of assembling such medical devices.

### BACKGROUND

Elongated medical devices, such as endoscopes and bronchoscopes, generally include a flexible shaft, a working distal tip, and a flexible steerable shaft joining the working tip and the flexible shaft. The flexible steerable shaft may include a bendable articulation joint and steering wires operable to bend the articulation joint. Often, the steering wires may be threaded through the articulation joint, which may increase assembly difficulty or reduce working channel space.

Additionally, many articulation joints are comprised of a single material and have a single stiffness that is not easily modifiable for various different devices. In other words, the amount of effort required to articulate the joint and resultant clinical performance is constant for a given articulation joint design, so that the design may not be suitable for different devices or procedures. However, certain medical devices, procedures, and/or users may benefit from more tactile feedback and, accordingly, more stiffness. Similarly, some medical devices, procedures, and/or users may prefer less stiffness. This disclosure may solve one or more of these problems or other problems in the art. The scope of the disclosure, however, is defined by the attached claims and not the ability to solve a specific problem.

### SUMMARY OF THE DISCLOSURE

According to an example, an articulation joint for a medical device may have a proximal end and a distal end. The articulation joint may include a plurality of links, one or more actuating wire, and at least one stiffening element. Each link of the plurality of links may be configured to articulate relative to adjacent links of the plurality of links. Each actuating wire may extend through an actuation channel of the plurality of links. Each actuating wire may be fixedly attached proximate the distal end of the articulation joint and may be translatable relative to the plurality of links to articulate the articulation joint. The at least one stiffening element may extend through a stiffening channel of the plurality of links. The at least one stiffening element may have a proximal end fixedly attached to one link of the plurality of links and may have a distal end fixedly attached to another link of the plurality of links.

In other examples, each link of the plurality of links may include a wall having a radially inner surface and a radially outer surface, wherein each stiffening channel is defined by the radially outer surface of a corresponding link and is open to a side of the corresponding link. Each link of the plurality of links may include a wall having a radially inner surface and a radially outer surface, wherein each actuation channel is defined by the radially outer surface of a corresponding link and is open to a side of the corresponding link. The stiffening channel may have a concave shape. The stiffening channel may be enclosed by a wall of the corresponding link. The actuation channel may have an enclosed shape. The stiffening channel may have a rectangular cross sectional shape. The at least one stiffening element may include a first stiffening element and a second stiffening element disposed opposite the first stiffening element. The plurality of links may include a particular link, a proximal link, and a distal link, wherein the at least one stiffening element comprises two proximal stiffening elements that couple the particular link to the proximal link and two distal stiffening elements that couple the particular link to the distal link. The two proximal stiffening elements may be arranged opposite each other and 90 degrees to each of the two distal stiffening elements. The at least one stiffening element may be continuous from the proximal end to the distal end. The one or more actuating wires may include two or four actuating wires. The articulation joint may include gaps disposed between the links of the plurality of links, wherein the gaps are narrowest at a location of the at least one stiffening element. The articulation joint may include a second stiffening element. Each link of the plurality of links may include a wall having a radially inner surface and a radially outer surface, wherein each stiffening channel is disposed on the radially outer surface of a corresponding link.

In other examples, a method of assembling a medical device may include selecting a stiffening element, assembling the stiffening element into stiffening channels of links of an articulation joint of the medial device, and fixedly attaching a proximal end of the stiffening element to one link of the links and a distal end of the stiffening element to another link of the links. The articulation joint may include at least one actuating wire extending through actuation channels of the links such that actuation of the at least one actuating wire causes each link of the links to articulate relative to an adjacent link of the links.

In other examples, each link of the links may include a wall having a radially inner surface and a radially outer surface, wherein each stiffening channel of the stiffening channels is defined by the radially outer surface of a corresponding link and is open to a side of the corresponding link.

In other examples, a medical device may include a handle, an articulation joint, a flexible shaft disposed between the handle and the articulation joint, and a tip disposed distally of the articulation joint. The articulation joint may have a proximal end and a distal end and may include a plurality of links, one or more actuating wires, and at least one stiffening element. Each link of the plurality of links may be configured to articulate relative to adjacent links of the plurality of links. Each actuating wire may extend through an actuation channel of each link of the plurality of links and may be fixedly attached proximate the distal end of the articulation joint and translatable relative to the plurality of links to articulate the articulation joint. The at least one stiffening element may extend through a stiffening channel of each link of the plurality of links. The at least one stiffening element may have a proximal end fixedly attached to one link of the plurality of links and may have a distal end fixedly attached to another link of the plurality of links.

In other examples, each link may define four stiffening channels. The stiffening element may be nonmetallic.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a side view of a medical device, according to an embodiment;

FIG. 2A is an end view of a link of an articulation joint of the medical device of FIG. 1, according to an embodiment;

FIG. 2B is a perspective view of another link of an articulation joint, according to an embodiment;

FIG. 2C is an end view of yet another link of an articulation joint, according to an embodiment;

FIG. 3 is a perspective view of the articulation joint having links of FIG. 2A, according to an embodiment;

FIG. 4A is a perspective view of another articulation joint, according to an embodiment;

FIG. 4B is another perspective view of the articulation joint of FIG. 4A, according to an embodiment;

FIG. 5A is a perspective view of another link of an articulation joint, according to an embodiment; and

FIGS. 5B and 5C are exploded and assembled perspective views, respectively, of an articulation joint having links of FIG. 5A, according to an embodiment.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

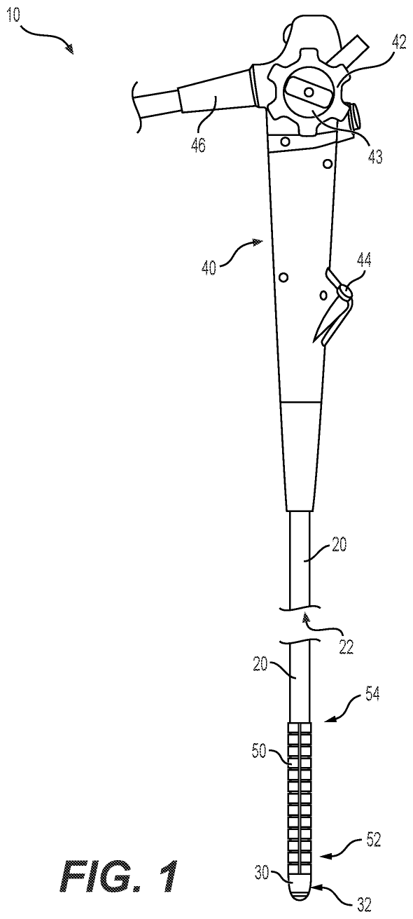

Referring to FIG. 1, a medical device 10 according to an embodiment is shown. The medical device 10 may be an endoscope, bronchoscope, ureteroscope, colonoscope, duodenoscope, cystoscope, catheter, sheath, or other elongated device for insertion into a patient. The medical device 10 includes a flexible shaft 20, a tip 30 at a distal end 32, and an articulation joint 50 disposed between and connecting the flexible shaft 20 and the tip 30. A handle 40 or some other device for actuating or controlling the medical device 10 and any tool or devices associated with the medical device 10 is connected at a proximal end of the flexible shaft 20.

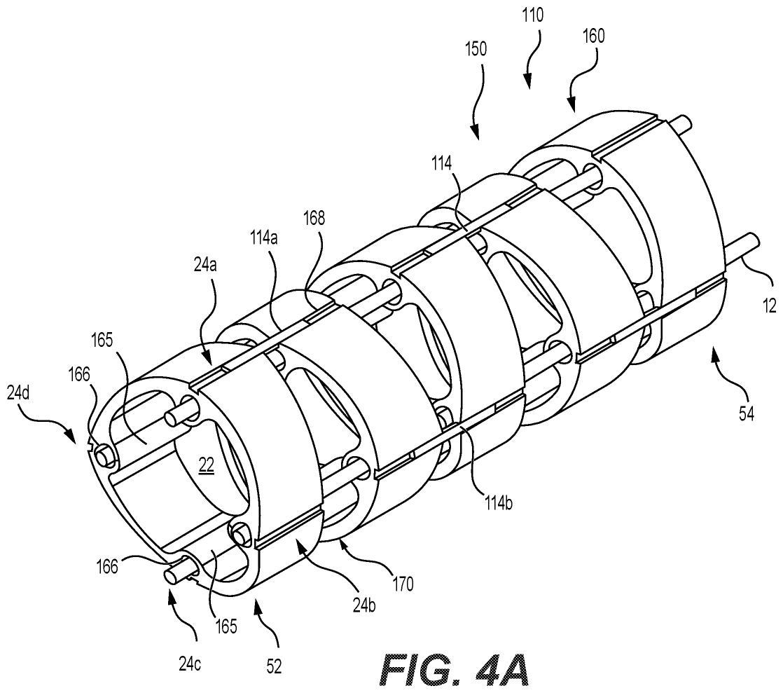

Referring to FIG. 1 in combination with FIG. 3, one or more actuating elements 12 may extend distally from a proximal end of the medical device 10. The actuation elements 12 may be any appropriate structure and material, such as cables or wires suitable for medical procedures (e.g., medical grade plastic or metal). The actuating elements 12 may extend into handle 40 and may be indirectly coupled to an actuating device 42, which controls articulation of articulation joint 50. The actuating device, may be, for example, a rotatable knob that rotates about an axis to push/pull and apply tension to actuating elements 12. Alternatively, or additionally, a user may operate the actuating elements 12 independently of handle 40. A locking knob 43 may be used to hold, or lock, actuating device 42 in a desired actuated position, thereby maintaining articulation joint 50 in a desired bent orientation.

The actuating elements 12 extend through flexible shaft 20 and terminate at articulation joint 50 (at or near a distal end 52 of articulation joint 50) and/or tip 30. For example, one or more actuating elements 12 may be connected to articulation joint 50 and/or one or more other actuating elements 12 may be attached to tip 30. As will be explained herein, actuation (push/pull; tensioning) of actuating elements 12 may control bending of articulation joint 50. In addition, one or more electrical cables (not shown) may extend from the proximal end of medical device 10 to tip 30 and may provide electrical controls to imaging, lighting, and/or other electrical devices on tip 30, and may carry imaging signals from tip 30 proximally to be processed and/or displayed on a display. Handle 40 may also include ports 44, 46 for introducing and/or removing tools, fluids, or other materials from the subject. Port 44 may be used to introduce tools. Port 46 may be connected to an umbilicus for introducing fluid, suction, and/or wiring for electronic components.

Referring still to FIGS. 1 and 3, one or more stiffness elements 14 may extend through, or adjacent to, the articulation joint 50. The stiffness elements 14 may extend continuously from a proximal end 54 of the articulation joint 50 to the distal end 52 of the articulation joint 50. The stiffening elements 14 may be coupled to the proximal end 54 of the articulation joint 50 at a first attachment point and may be coupled to the distal end 52 at a second attachment point. The stiffness elements 14 may be any appropriate structure and material, such as cables or wires suitable for medical procedures (e.g., medical grade plastic or metal). In some embodiments, the stiffness elements 14 may be the same material and/or structure as the actuating elements 12, while in other embodiments, the materials and/or structure may differ. In particular, in some embodiments, the stiffness elements 14 may be a stiffer material and structure than the actuating elements 12. This may provide increased stiffness to the articulation joint 50 while still allowing bending or movement of the articulation joint 50. In other embodiments, the stiffness elements 14 may be a less stiff material and structure than the actuating elements 12. This may provide a relatively less stiff articulation joint 50. In still other embodiments, a material, a stiffness, a cross-sectional size, and/or a length of one stiffness element 14 may differ from that of another stiffness element 14 of the same articulation joint 50. In some embodiments, the stiffness elements 14 may be a different material than the material of the links 60, which are described further herein. Suitable materials for a stiffness element 14 include stainless steel, nitinol, or other metallic or nonmetallic materials, such as resin or plastic.

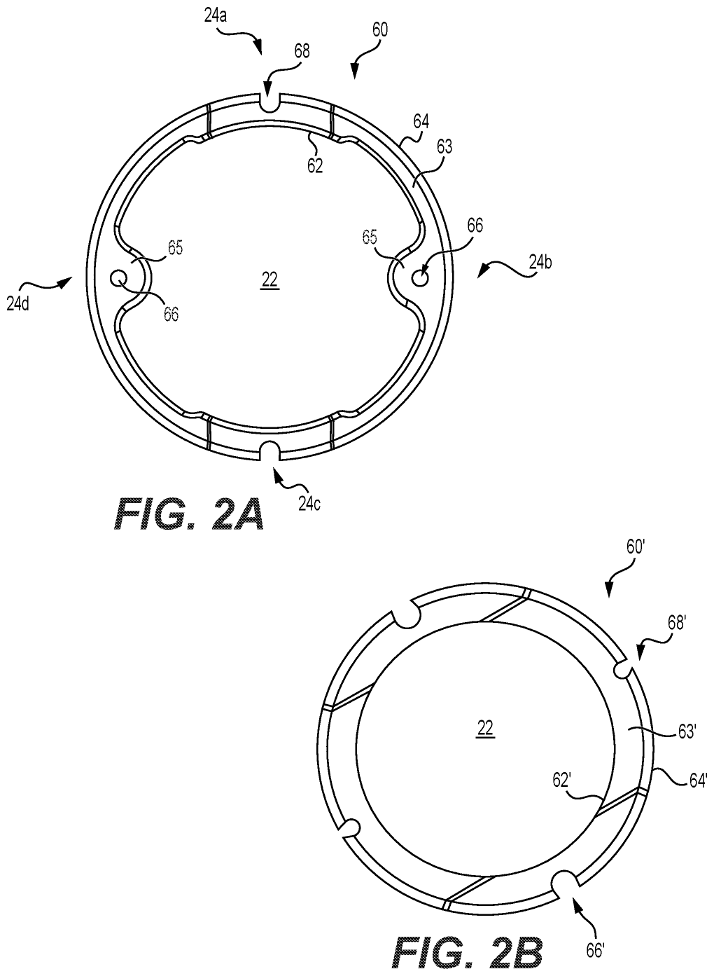

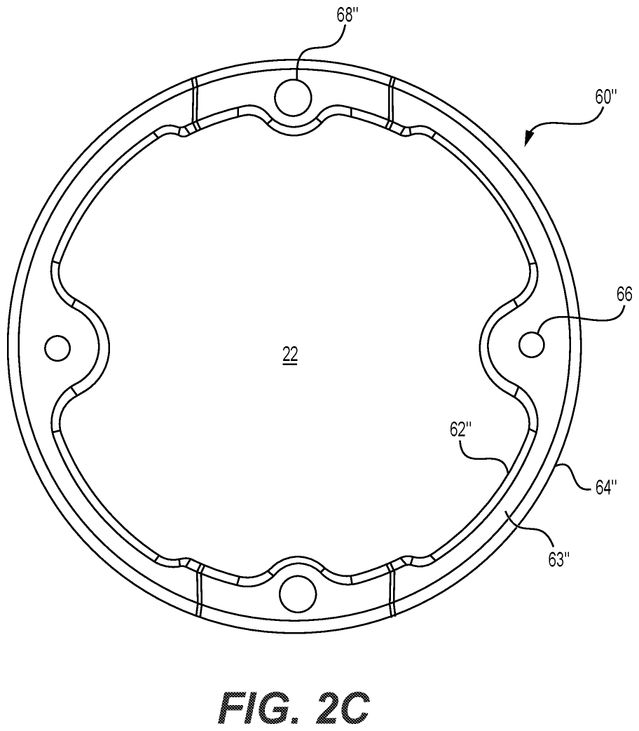

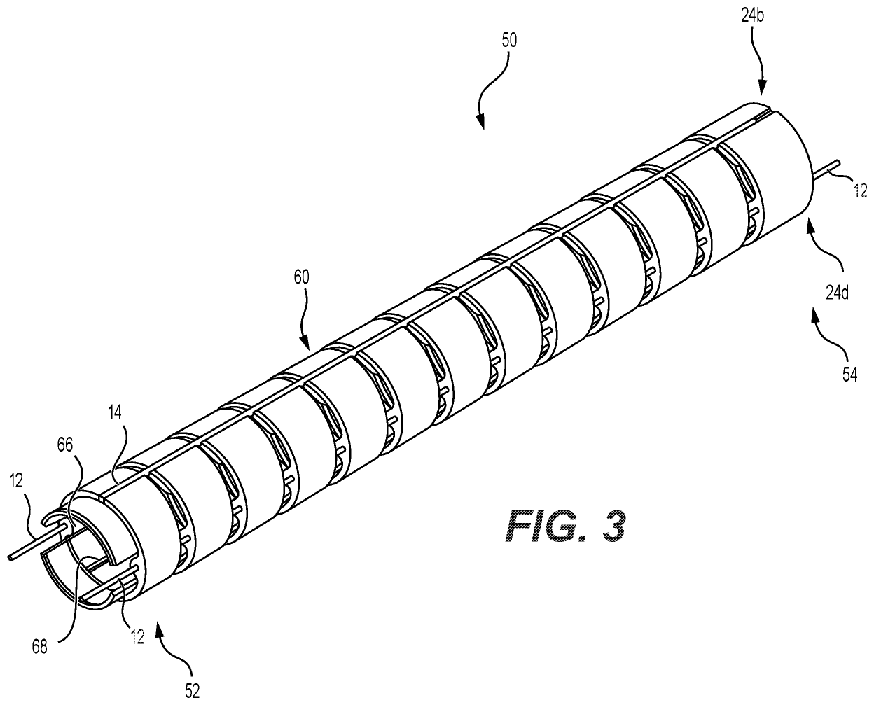

As shown in FIGS. 1-2C, articulation joint 50 includes a portion of a lumen 22 that extends through medical device 10. The lumen 22 may extend from the handle 40 through the flexible shaft 20 into the articulation joint 50, and through a distal end of tip 30. The lumen 22 may receive tools, imaging devices, and other devices associated with the medical device 10 to perform endoscopic or other medical procedures. Further, tissue samples and/or other material may be removed from a subject through the lumen 22. It will be understood that the medical device 10, including flexible shaft 20 and articulation joint 50, are not limited to a single channel/lumen 22, and may include any number of lumens necessary for performing procedures. Alternatively, or additionally, one or more catheters (not shown) may be introduced through the lumen 22 to remove tissue and/or insert tools.

With reference to a first embodiment shown in FIGS. 1, 2A, and 3, the articulation joint 50 may include links 60 arranged along an axis of articulation joint 50. Each link 60 may have an inner surface 62 and an outer surface 64 defining a wall 63 therebetween. The lumen 22 may extend through link 60 and be defined by the inner surface 62. The wall 63 of each of the links 60 may define protrusions 65 extending radially inwardly into channel/lumen 22. Each protrusion 65 includes and fully encloses an actuation channel 66 through which an actuating element 12 may extend. In some embodiments, each link 60 may have two actuation channels 66 positioned opposite each other. In other words, the actuation channels 66 may be spaced apart by approximately 180 degrees about the inner surface 62.

Each link 60 may define stiffening channels 68 through which the stiffness elements 14 may extend. The stiffening channels 68 may be recesses within the wall 63 of the link 60 and may have a concave shape such as a curved or polygonal shape oriented inward and into the wall 63. The stiffening channels may be disposed on and defined by the outer surface 64, be open to a side of the link 60, and not be fully enclosed by the wall 63 of the link 60. In some embodiments, each link 60 may have two or more stiffening channels 68 positioned radially about the outer surface. In particular, in some embodiments, each link 60 may have two stiffening channels 68 positioned opposite each other. In other words, the stiffening channels 68 may be spaced apart by approximately 180 degrees about the outer surface 64.

FIG. 2B shows another embodiment of a link 60′ for use in an articulation joint 50. Like reference numerals will be used to de-scribe link 60′. Link 60′ is similar to link 60 except as described herein. For example, lumen 22 may extend through link 60′ and be defined by inner surface 62′. As depicted, the actuation channels 66′ may be disposed on the outer surface 64′ and may not be fully enclosed by the wall 63′ of the link 60′, like stiffening channels 68 of link 60 in FIG. 2A. In other words, the actuation channels 66′ may be concave, U-shaped recesses within the wall 63′ of the link 60′. The actuation channels 66′ may be disposed on and defined by the outer surface 64, be open to a side of the link 60′, and not be fully enclosed by the wall 63′ of the link 60′. Relative to the link 60 in FIG. 2A, this may provide a better line of sight to the actuation channels 66′, which may allow the actuation elements 12 to be more easily assembled within the links 60′. In some embodiments, the actuation channels 66′ may have the same geometry and cross-sectional shape as the stiffening channels 68′. In other embodiments, the respective geometries and cross-sectional shapes may differ. For example, as depicted in FIG. 2B, the stiffening channels 68′ may be smaller than the actuation channels 66′. This may account for differences in the sizes of the actuating elements 12 and the stiffening elements 14.

FIG. 2C shows another embodiment of a link 60″ for use in an articulation joint 50. Like reference numerals will be used to describe link 60″. Link 60″ is similar to link 60 except as described herein. In particular, the stiffening channels 68″ may be fully enclosed within the wall 63″ of the link 60″, like actuation channels 66 of the link 60. Accordingly, in some embodiments, both the stiffening channels 68″ and the actuation channels 66 may be fully enclosed by the wall 63″ and also may have differing cross-sectional sizes, as shown in FIG. 2C. Lumen 22 may extend through link 60″ and be defined by inner surface 62″. In some embodiments, each actuation channel 66 and each stiffening channel 68″ of link 60″ may be disposed between and fully enclosed by both inner surface 62″ and outer surface 64″.

In light of FIGS. 1-3, the stiffness of the articulation joint 50, both in-plane and out of plane, may be varied by varying the stiffness of the stiffness elements 14. Accordingly, the medical device 10 may be customizable with respect to stiffness of the articulation joint 50 by selection of the stiffness elements 14 and without alteration of other components.

A method of assembling the medical device 10 may include selecting the stiffening element 14 based on a selection criteria such as user stiffness preference or the procedure for which use of the medical device 10 is intended. For example, in some embodiments, a method of assembly may include collecting stiffness preferences from users of a particular kind of medical device or that perform a particular type of medical procedure. The method may further include then selecting the corresponding stiffening element 14 and assembling the selected stiffening element 14 into the medical device 10. In this manner, various medical devices sharing the articulation joint design can have articulation joints of varying stiffnesses. In embodiments, different types of medical devices can have articulation joints of varying stiffnesses, and in other embodiments, the same type of medical device may be made available with a variety of articulation joints of different stiffnesses, so that a user may choose a preferred articulation joint stiffness.

With continued reference to FIGS. 1-3, a method of assembling the medical device 10 may include assembling the stiffness elements 14 within the stiffening channels 68 of the medical device 10. In particular, in some embodiments, the stiffness elements 14 may first be manufactured as a wire shape and may then be inserted or threaded into the stiffening channels 68. The stiffness elements 14 may then be nonmoveably coupled to the links 60. For example, the stiffness elements 14 may be brazed, welded or adhered with adhesive to one or more links 60. Alternatively or additionally, stiffness elements 14 may be coupled with an interference fit within the stiffening channels 68. In some embodiments, the stiffness elements 14 may be nonmoveably coupled at each link 60 and/or at each of the stiffening channels 68.

In other embodiments, the stiffness elements 14 may not be first manufactured as a wire, coil, or braid. Instead, a method of assembling the medical device 10 may include molding the stiffness elements 14 in place within the stiffening channels 68. In particular, such a method may include inserting a core into the lumen 22 and about the outer surfaces 64 of the links 60 such that a fluid material (e.g., molten metal or nonmetallic material such as plastic or uncured resin) may be flowed through the stiffening channels 68 and cured or solidified in place.

In light of FIGS. 1-3, a method of operating the medical device 10 may include inserting the medical device 10 into a patient. The method may then include actuating/bending the articulation joint 50 such that the tip 30 may be moved to access or view a space within the patient. More specifically, the method may include tightening and/or loosening the actuation elements 12 to actuate the articulation joint 50. For example, one of the actuation elements 12 may be tightened or pulled tight on a second side 24b while a second of the actuation elements 12 may be loosened on a fourth side 24d that is opposite the second side 24b. This may cause the articulation joint 50 to bend toward the second side 24b. Because the articulation joint 50 has two actuation elements 12, the articulation joint 50 may be moveable in two directions, i.e. toward the first side 24a and toward the third side 24c. These two directions are in the same plane.

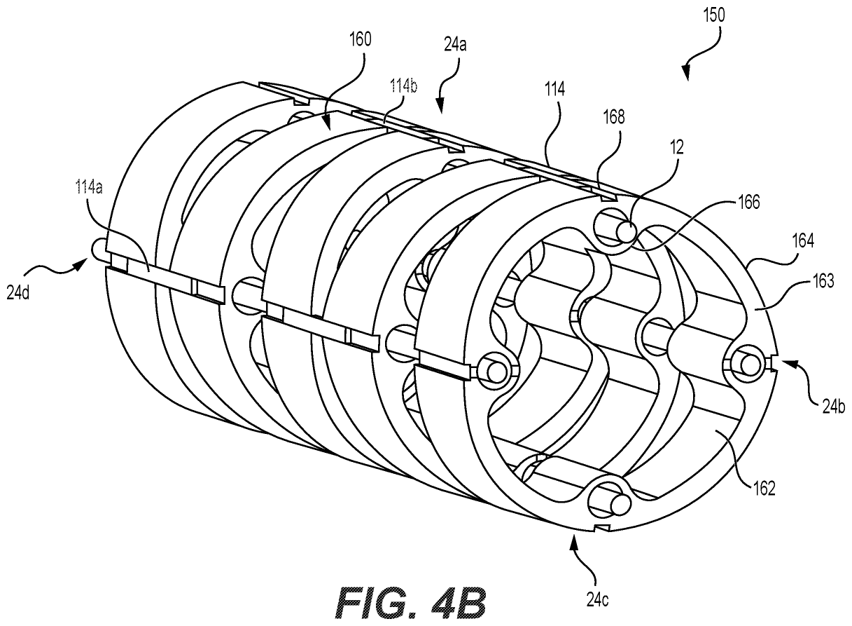

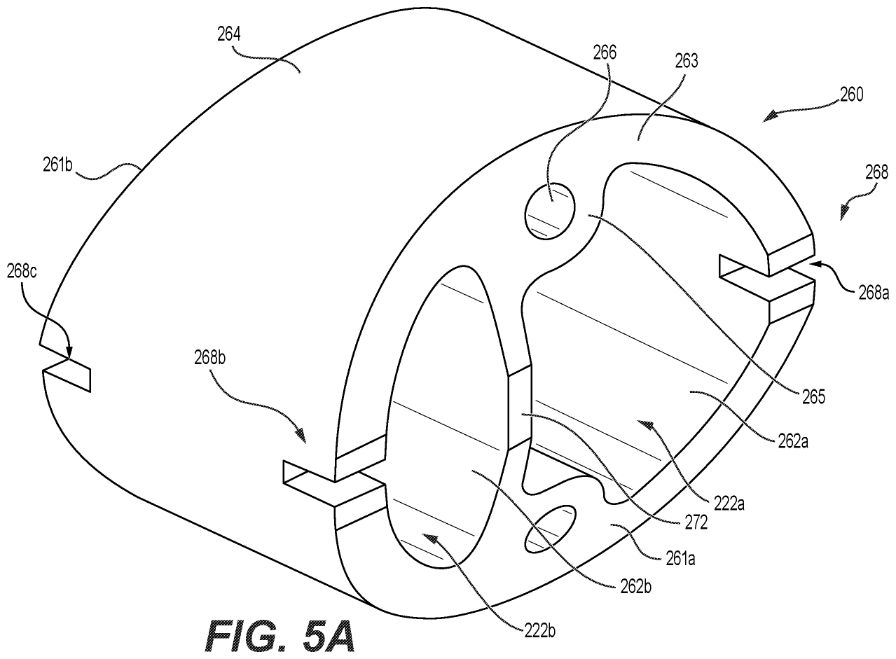

Referring now to FIGS. 4A and 4B, an embodiment of an articulation joint 150 for a medical device 110 is schematically depicted. The articulation joint 150 may be substantially similar to the articulation joint 50. Accordingly, like numbers will be used to refer to like features, with “100” added to each numeral. For example, the articulation joint 150 may include links 160, and each link of the links 160 may have an inner surface 162, an outer surface 164, and a wall 163 therebetween.

Each of the links 160 may define actuation channels 166 through which actuation elements 12 may extend. The wall 163 of each of the links 160 may define four protrusions 165 extending radially inwardly into channel/lumen 22. Each protrusion 165 includes and fully encloses an actuation channel 166 through which an actuating element 12 may extend. In some embodiments, each of the links 160 may have four actuation channels 166 spaced 90 degrees from one another, such as depicted. However, other embodiments may have other quantities of actuation channels, such as one channel, two channels, or three channels.

The articulation joint 150 may have four actuation elements 12 extending through the actuation channels 166. Accordingly, the articulation joint 150 may be moveable/bendable in four directions, e.g. toward the first side 24a, toward the second side 24b, toward the third side 24c, and toward the fourth side 24d. The articulation joint 150 therefore has four-way bending in two transverse planes, whereas other embodiments described herein are limited to two-way bending in one plane. As will be appreciated by those skilled in the art, a medical device having the articulation joint 150 may include a second actuating device (similar to the actuating device 42 depicted in FIG. 1) to control the movement of the third and fourth actuation elements 12.

With continued reference to FIGS. 4A and 4B, each link 160 may define stiffening channels 168 through which stiffness elements 114 may extend. The stiffening channels 168 may have a concave or enclosed geometry and may be disposed on and defined by the outer surface 164. More specifically, each stiffening channel 168 may have a concave geometry with an approximately rectangular cross sectional shape, such as depicted. Each stiffening channel 168 may be open to a side of the link 160 and not be fully enclosed by the wall 163 of the link 160. In some embodiments, each link 160 may have four stiffening channels 168. Each stiffening channel 168 is located radially outside of a corresponding actuation channel 166. The stiffness channels 168 may be evenly distributed about the outer surface 164. For example, in embodiments including four stiffening channels 168, the stiffening channels 168 may be spaced apart by approximately 90 degrees. Other quantities and locations of stiffening channels are contemplated and possible.

The articulation joint 150 may have a plurality of stiffness elements 114. Each stiffness element 114 may couple one of the links 160 to another of an adjacent link or multiple links. In particular, in some embodiments, at least one stiffness element 114 may couple a particular link 160 to only a proximal adjacent link 160, and at least another stiffness element 114 may couple the particular link 160 to only a distal adjacent link 160.

In some embodiments, each link 160 may be coupled to four stiffness elements 114. In such embodiments, two proximal stiffness elements 114a may couple a particular link 160 to a proximal adjacent link 160, and another two distal stiffness elements 114b may couple the particular link 160 to a distal adjacent link 160. The two proximal stiffness elements 114a may be arranged opposite each other, i.e. at approximately 180 degrees from each other. Similarly, the two distal stiffness elements 114b may be arranged opposite each other, and 90 degrees from the proximal stiffness elements 114a. As depicted, the stiffness elements 114 may be discontinuous, or spaced apart, between the distal end 52 and the proximal end 54.

Disposed between the links 160 may be gaps 170. Each gap 170 may vary in length between axially opposed points on adjacent links 160 and specifically between opposing edges of the outer surfaces 164 of those adjacent links 160. About the circumference of adjacent links 160, the gaps 170 may be narrowest at the position of stiffness elements 114 and may be widest between the stiffness elements 114, for example at the midpoint between, and 90 degrees from, stiffness elements 114. As described above, the two distal stiffness elements 114b may be arranged opposite each other, and 90 degrees from the proximal stiffness elements 114a. Accordingly, the widest part of the gaps 170 at a distal end of a particular link 160 may be arranged opposite each other and 90 degrees from the widest part of the gaps 170 at a proximal end of the link 160. This arrangement of stiffness elements and gaps enables bending of the articulation joint 150 in four directions, including towards the locations of the stiffness elements 114.

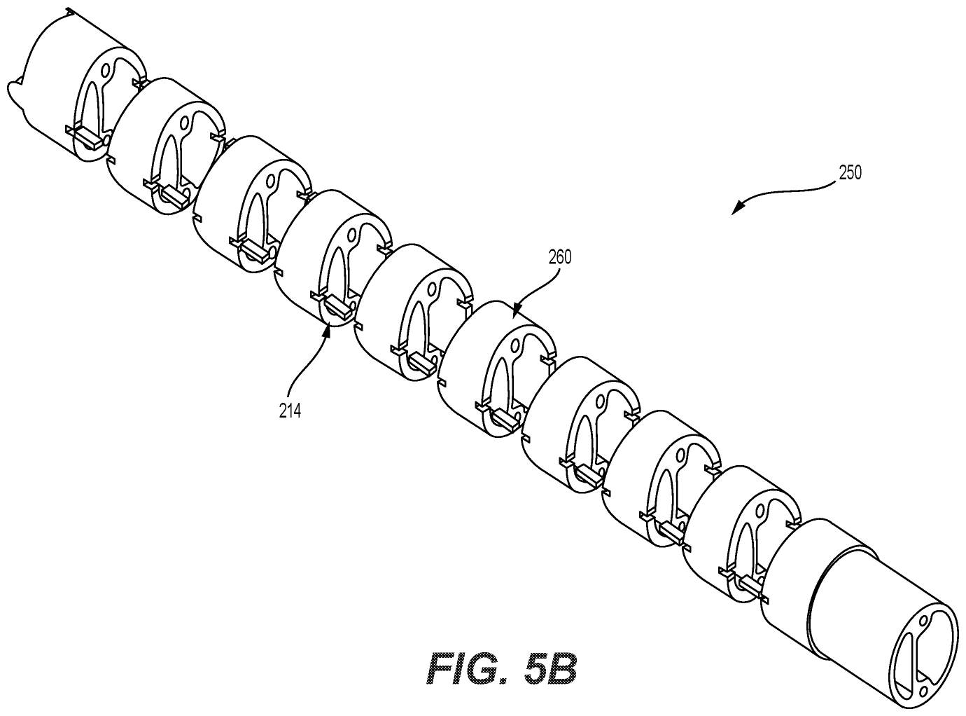

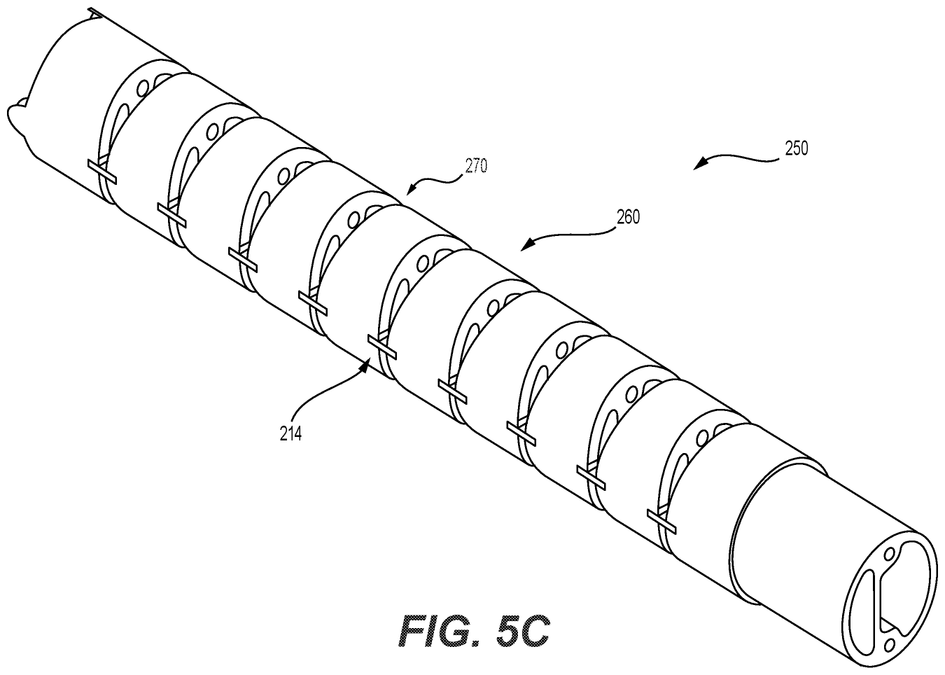

Referring now to FIGS. 5A-5C, an embodiment of an articulation joint 250 for a medical device is schematically depicted. The articulation joint 250 may be substantially similar to the articulation joints 50 and 150. Accordingly, like numbers will be used to refer to like features, with another “100” added to the numeral. The articulation joint 250 may include a plurality of links 260, and each of the links 260 may have an outer surface 264, a first inner surface 262a, a second inner surface 262b, and a wall 263 having a front (distal) surface 261a, and a rear (proximal) surface 261b. Extending through each of the links 260 may be a first lumen 222a and a second lumen 222b. The first lumen 222a may be defined by the first inner surface 262a, and the second lumen 222b may be defined by the second inner surface 262b. A wall 272 separates the first lumen 222a from the second lumen 222b. The first lumen 222a may be a working channel to accommodate tools or other devices inserted through the port 44, and the second lumen 222b may accommodate wires/cables for sending power to distal tip 30 and/or sending/receiving imaging signals/data to and from the distal tip 30.

Each of the links 260 may define protrusions 265 extending radially inwardly towards the lumen 222a. In other embodiments, the protrusions 265 may extend radially inwardly towards the lumen 222b. Each protrusion 265 includes and fully encloses an actuation channel 266 through which an actuating element 12 may extend. In some embodiments, each link 260 may have two actuation channels 266 positioned opposite each other. In other words, the actuation channels 266 may be spaced apart by approximately 180 degrees about wall 263.

Each link 260 may define stiffening channels 268 through which the stiffness elements 214 may extend. The stiffening channels 268 may have a concave geometry and may extend axially from the front surface 261a and the rear surface 261b into the wall 263 of the link 260. The stiffening channels 268 may extend entirely through a radial thickness of the wall 263 of the link 260, i.e. from the outer surface 264 to the first inner surface 262a and/or the second inner surface 262b. As depicted, in some embodiments, each link 260 may have two stiffening channels 268 at the front surface 261a and two stiffening channels at the rear surface 261b. A first stiffening channel 268a may extend from the outer surface 264 to the first inner surface 262a, and a second stiffening channel 268b may extend from the outer surface 264 to the second inner surface 262b. In some embodiments, the first stiffening channel 268a may be positioned opposite the second stiffening channel 268b. In other words, the stiffening channels 268a, 268b may be spaced apart by approximately 180 degrees about the outer surface 264. Two like stiffening channels 268 are at the rear surface 261b and are axially aligned with the stiffening channels 268a, 268b (see stiffening channel 268c in FIG. 5A and another stiffening channel not shown in FIG. 5A as it is blocked by structure of link 260). The stiffening channels 268 may have a substantially rectangular cross sectional shape, such as depicted.

Accordingly, each stiffening channel 268 may accommodate a stiffening element 214, such as depicted in FIGS. 5B and 5C. Each stiffening element 214 has a rectangular cross section (taken in both an axially directed plane and a width/radially directed plane transverse to the axial plane). Each stiffening element 214 connects two adjacent links 260. Each stiffening element 214 may be adhered to its corresponding stiffening channel 268 via any suitable method, including those mentioned in connection with above-described embodiments.

Disposed between the links 260 may be gaps 270. Gaps 270 may have the same or similar shape and configuration as gaps 170 of FIGS. 4A and 4B. The gaps 270 may be narrowest at the stiffness elements 214 and may be widest about the circumference of the links 260 between the stiffness elements 214. This arrangement may allow bending of the articulation joint 250 in two directions.

It will be apparent to those skilled in the art that various modifications and variations can be made to the disclosed device without departing from the scope of the disclosure. For examples, the number, configuration, shape, and/or spacing of actuation channels and/or stiffening channels may be altered to suit any medical device. It will be understood that the links, and their corresponding stiffening and actuation channels and stiffening elements are not limited to the examples described herein. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An articulation joint for a medical device, the articulation joint having a proximal end and a distal end and comprising:
a plurality of links, each link of the plurality of links configured to articulate relative to adjacent links of the plurality of links;
one or more actuating wires, each actuating wire extending through an actuation channel of each link of the plurality of links, fixedly attached proximate the distal end of the articulation joint, and translatable relative to the plurality of links to articulate the articulation joint; and
at least one stiffening element extending through a stiffening channel of each link of the plurality of links, having a proximal end fixedly attached to one link of the plurality of links, and having a distal end fixedly attached to another link of the plurality of links.

2. The articulation joint of claim 1, wherein each link of the plurality of links includes a wall having a radially inner surface and a radially outer surface, wherein each stiffening channel is defined by the radially outer surface of a corresponding link and is open to a side of the corresponding link.

3. The articulation joint of claim 2, wherein each link of the plurality of links includes a wall having a radially inner surface and a radially outer surface, wherein each actuation channel is defined by the radially outer surface of a corresponding link and is open to a side of the corresponding link.

4. The articulation joint of claim 1, wherein the stiffening channel has a concave shape.

5. The articulation joint of claim 2, wherein the stiffening channel is enclosed by a wall of the corresponding link.

6. The articulation joint of claim 1, wherein the actuation channel has an enclosed shape.

7. The articulation joint of claim 1, wherein the stiffening channel has a rectangular cross sectional shape.

8. The articulation joint of claim 1, wherein the at least one stiffening element includes a first stiffening element and a second stiffening element disposed opposite the first stiffening element.

9. The articulation joint of claim 1, wherein the plurality of links comprises a particular link, a proximal link, and a distal link, and wherein the at least one stiffening element comprises two proximal stiffening elements that couple the particular link to the proximal link and two distal stiffening elements that couple the particular link to the distal link.

10. The articulation joint of claim 9, wherein the two proximal stiffening elements are arranged opposite each other and 90 degrees to each of the two distal stiffening elements.

11. The articulation joint of claim 1, wherein at least one stiffening element is continuous from the proximal end to the distal end.

12. The articulation joint of claim 1, wherein the one or more actuating wires includes two or four actuating wires.

13. The articulation joint of claim 1, further comprising gaps disposed between each link of the plurality of links, wherein the gaps are narrowest at a location of the at least one stiffening element.

14. The articulation joint of claim 1, wherein the articulation joint further comprises a second stiffening element.

15. The articulation joint of claim 1, wherein each link of the plurality of links includes a wall having a radially inner surface and a radially outer surface, wherein each stiffening channel is disposed on the radially outer surface of a corresponding link.

16. A method of assembling a medical device comprising:
selecting a stiffening element;
assembling the stiffening element into stiffening channels of links of an articulation joint of the medical device; and
fixedly attaching a proximal end of the stiffening element to one link of the links, and a distal end of the stiffening element to another link of the links,
wherein the articulation joint includes at least one actuating wire extending through actuation channels of the links such that actuation of the at least one actuating wire causes each link of the links to articulate relative to an adjacent link of the links.

17. The method of claim 16, wherein each link of the links includes a wall having a radially inner surface and a radially outer surface, wherein each stiffening channel of the stiffening channels is defined by the radially outer surface of a corresponding link and is open to a side of the corresponding link.

18. A medical device comprising: handle, an articulation joint, a flexible shaft disposed between the handle and the articulation joint, and a tip disposed distally of the articulation joint, wherein the articulation joint has a proximal end and a distal end and comprises:
a plurality of links, each link of the plurality of links configured to articulate relative to adjacent links of the plurality of links;
one or more actuating wires, each actuating wire extending through an actuation channel of each link of the plurality of links, fixedly attached proximate the distal end of the articulation joint, and translatable relative to the plurality of links to articulate the articulation joint; and
at least one stiffening element extending through a stiffening channel of each link of the plurality of links, having a proximal end fixedly attached to one link of the plurality of links, and having a distal end fixedly attached to another link of the plurality of links.

19. The articulation joint of claim 18, wherein each link defines four stiffening channels.

20. The articulation joint of claim 18, wherein stiffening element is nonmetallic.
